(19) United States
(12) Patent Application Publication
(10) Pub. No.: US 2016/0199364 A1
(43) Pub. Date: Jul. 14, 2016

(54) SEIZURE TREATMENT COMPOSITIONS AND METHODS

(71) Applicant: Creighton University, Omaha, NE (US)

(72) Inventors: Timothy A. Simeone, Omaha, NE (US); Kristina A. Simeone, Omaha, NE (US)

(21) Appl. No.: 14/994,320

(22) Filed: Jan. 13, 2016

Related U.S. Application Data
(60) Provisional application No. 62/102,681, filed on Jan. 13, 2015.

(51) Int. Cl. A61K 31/4439; A61K 9/00; A61K 31/167

(52) U.S. Cl. CPC A61K 31/4439; A61K 31/167; A61K 9/0053; A61K 9/0019; A23L 1/296; A23V 2002/00

(57) ABSTRACT

A composition, a method for administering an anti-seizure composition, and a method for treating a seizure are described. In an implementation, a composition comprises a peroxisome proliferator activated receptor gamma (PPARγ) antagonist that is administered to an individual, where the individual has been administered a ketogenic diet. In an implementation, a method for administering an anti-seizure composition comprises administering a ketogenic diet to an individual; and concurrently administering a therapeutically effective dose of a peroxisome proliferator activated receptor gamma (PPARγ) antagonist to the individual In an implementation, a method for treating a seizure comprises administering a therapeutically effective dose of a peroxisome proliferator activated receptor gamma (PPARγ) antagonist to an individual on a ketogenic diet and suffering from seizures.

### CROSS-REFERENCE TO RELATED APPLICATIONS

The present application claims the benefit under 35 U.S.C. §119(e) of U.S. Provisional Application Ser. No. 62/102,681, filed Jan. 13, 2015, and titled “SEIZURE TREATMENT COMPOSITIONS AND METHODS.” U.S. Provisional Application Ser. No. 62/102,681 is herein incorporated by reference in its entirety.

### BACKGROUND

Epilepsy is a group of neurological disorders in which nerve cell activity in the brain becomes disrupted and is characterized by epileptic seizures, which are episodes that include seizures from brief and nearly undetectable to long periods of vigorous shaking. Epileptic seizures tend to recur and have no immediate underlying cause and are often associated with cell toxicity, hyperexcitability and death. It is estimated that about 1% of people worldwide have epilepsy.

Epilepsy drugs can be prescribed to control seizures. Seizures can be controlled with medication in about 70-80% of cases. When medication is not effective, other treatment considerations may include surgery, neurostimulation, and/or dietary changes.

### SUMMARY

A composition, a method for administering an anti-seizure composition, and a method for treating a seizure are described. In an implementation, a composition comprises a peroxisome proliferator activated receptor gamma (PPARγ) antagonist that is administered to an individual, where the individual has been administered a ketogenic diet. In an implementation, a method for administering an anti-seizure composition comprises administering a ketogenic diet to an individual; and concurrently administering a therapeutically effective dose of a peroxisome proliferator activated receptor gamma (PPARγ) antagonist to the individual In an implementation, a method for treating a seizure comprises administering a therapeutically effective dose of a peroxisome proliferator activated receptor gamma (PPARγ) antagonist to an individual on a ketogenic diet and suffering from seizures.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The detailed description is described with reference to the accompanying figures. The use of the same reference numbers in different instances in the description and the figures may indicate similar or identical items.

FIG. 1 is a graphical illustration depicting that a Ketogenic diet treatment does not change nuclear expression of PPARα in WT or Kv1.1KO brains (n=3-4 mice).

FIG. 2A illustrates a graphical depiction of the significance of PPARγ2 in KD anti-seizure efficacy. Effect of genotype, dietary treatments and PPARγ antagonism (GW9662) on brain cell nuclear expression of PPARγ2/γ1 ratio (n=4-10 mice; two-way analysis of variance (ANOVA), interaction: F_{(3,40)}=3.003, P<0.05, treatment: F_{(3,40)}=5.278, P<0.01, genotype: F_{(1,40)}=15.05, P<0.001; ***P<0.001 as compared to Kv1.1 knockout (KO) fed a standard diet (SD) (Tukey's multiple comparisons post-hoc test)).

FIG. 2B illustrates a graphical depiction of the significance of PPARγ2 in KD anti-seizure efficacy. Effect of genotype, dietary treatments and PPARγ antagonism (GW9662) on brain cell nuclear expression of PPARγ2/β-actin ratio (n=4-10 mice; two-way ANOVA, treatment: F_{(3,40)}=8.976, P<0.001, genotype: F_{(1,40)}=2.619, P>0.05; *P<0.05, ***P<0.001 as compared to mice fed an SD within genotype (Tukey's multiple comparisons post-hoc test)).

FIG. 2C illustrates a graphical depiction of the significance of PPARγ2 in KD anti-seizure efficacy. Effect of genotype, dietary treatments and PPARγ antagonism (GW9662) on brain cell nuclear expression of PPARγ1/β-actin ratio (n=4-10 mice; two-way ANOVA, genotype: F_{(1,40)}=14.55, P<0.001; *P<0.05 as compared to WT-SD mice (Tukey's multiple comparisons post-hoc test)) normalized to samples from WT mice fed a SD.

FIG. 2D illustrates a representative EEG example of a generalized tonic-clonic seizure (modified Racine Scale 5) in a Kv1.1KO mouse fed a SD and corresponding time-frequency map constructed with a short-time Fourier transform.

FIG. 2E illustrates a graphical depiction of the effect of genotype, dietary treatments and PPARγ antagonism on seizure burden in WT and epileptic KO mice (n=4 mice; two-way ANOVA, interaction: F_{(3,23)}=4.986, P<0.01, treatment: F_{(3,23)}=4.986, P<0.01, genotype: F_{(1,23)}=68.96, P<0.0001; ***P<0.001 as compared to Kv1.1KO mouse fed a SD (Tukey's multiple comparisons post-hoc test).

FIG. 2F illustrates a graphical depiction of the effect of genotype, dietary treatments and PPARγ antagonism on seizure incidence in WT and epileptic Kv1.1KO mice (n=4; two-way ANOVA, interaction: F_{(3,23)}=4.986, P<0.01, treatment: F_{(3,23)}=4.986, P<0.01, genotype: F_{(1,23)}=68.96, P<0.0001). **P<0.01, *P<0.001 as compared to Kv1.1KO mouse fed a SD (Tukey's multiple comparisons post-hoc test).

FIG. 2G illustrates a graphical depiction of the effect of PPARγ antagonism on the latency to flurothyl-induced generalized tonic-clonic seizures of wild-type C3HeB/FeJ mice (n=6-7 mice).

FIG. 2H illustrates a graphical depiction of the calculated dosage of GW9662. Neither genotype nor treatment had a significant effect on water consumption (not shown) or resulting GW9662 dosage (n=4-6 mice).

FIG. 3A illustrates a graphical depiction of genetic knockout of PPARγ2 that eliminates KD-mediated seizure protection including latency to flurothyl-induced generalized tonic-clonic seizures of PPARγ2 wild-type (WT), heterozygous (HET) and knockout (KO) littermates fed a SD or KD (n=5-8 mice; two-way ANOVA, interaction: F_{(2,32)}=4.728, P<0.05, treatment: F_{(1,32)}=16.97, P<0.001). **P<0.01 as compared to SD (Tukey's multiple comparisons post-hoc test).

FIG. 3B illustrates a graphical depiction of genetic knockout of PPARγ2 that eliminates KD-mediated seizure protection including latency to flurothyl-induced generalized tonic-clonic seizures of foxed (fl/fl) control PPARγ^{fl/fl} mice and neuron-specific Synapsin I-Cre⁺ PPARγ^{fl/fl} knockout (NKO) mice fed a SD or KD (n=5-7; two-way ANOVA, interaction: F_{(1,19)}=6.588, P<0.05, treatment: F_{(1,19)}=16.43, P<0.001, genotype: F_{(1,19)}=7.805, P<0.05). **P<0.01 as compared to standard diet (SD) (Tukey's multiple comparisons post-hoc test).

FIG. 4A illustrates a graphical depiction of how pioglitazone mimics KD and the effect of a five-day pioglitazone (PIO; 10 mg/kg/day) treatment on brain cell nuclear expression of PPARγ1/β-actin ratio, PPARγ2/β-actin ratio and PPARγ2/γ1 ratio. *P<0.05, **P<0.01 as compared to Kv1.1KO (n=6-10 mice; Student's unpaired t-test).

FIG. 4B illustrates a graphical depiction of how pioglitazone mimics KD and the effect of a five-day treatment with pioglitazone (PIO; 10 mg/kg/day, i.p.) on Kv1.1KO seizure incidence compared to the average two-day vehicle baseline. *P<0.05, **P<0.01 as compared to vehicle (n=4 mice; repeated measures one-way ANOVA, treatment: F=7.972, P<0.01, individual: F=0.3991, P>0.05). *P<0.05. **P<0.01 as compared to vehicle (Tukey's multiple comparisons post-hoc test).

FIG. 4C illustrates a graphical depiction of how pioglitazone mimics KD and the effect of PIO treatment on Kv1.1KO seizure burden compared to the average two-day vehicle baseline (n=4 mice; repeated measures one-way ANOVA, treatment: F=9.472, P<0.01; *P<0.05, **P<0.01 as compared to vehicle (Tukey's multiple comparisons post-hoc test)).

FIG. 5A illustrates a graphical depiction showing where keone bodies, glucose and weight is unaffected by PPARγ modulation regardless of genotype or dietary treatment and the effect of genotype, dietary treatments and PPARγ antagonism on blood β-hydroxybutrate (n=4-6; two-way ANOVA, treatment: F_{(3,27)}=20.82, P<0.0001, genotype: F_{(1,27)}=1.759, P>0.05; *P<0.05, **P<0.01, ***P<0.001 as compared to SD (Tukey's multiple comparisons post-hoc test)).

FIG. 5B illustrates a graphical depiction showing where keone bodies, glucose and weight is unaffected by PPARγ modulation regardless of genotype or dietary treatment and the effect of genotype, dietary treatments and PPARγ antagonism on blood glucose (n=4-6; two-way ANOVA, treatment: F_{(3,27)}=9.46, P<0.001, genotype: F_{(1,27)}=9.923, P<0.01; **P<0.01 as compared to SD (Tukey's multiple comparisons post-hoc test)).

FIG. 5C illustrates a graphical depiction showing where keone bodies, glucose and weight is unaffected by PPARγ modulation regardless of genotype or dietary treatment and the effect of genotype, dietary treatments and PPARγ antagonism on body weight (n=4-6; two-way ANOVA, interaction: F=0.1806, P>0.05, treatment: F=8.211, P<0.001, genotype: F=15.83, P<0.001; **P<0.01 as compared to SD (Tukey's multiple comparisons post-hoc test)). Reported values are from postnatal day 35 mice.

FIG. 5D illustrates a graphical depiction showing that genetic loss of PPARγ does not alter blood β-hydroxybutrate (n=4-9 mice; two-way ANOVA, treatment: F=79.01, P<0.001; **P<0.01, ***P<0.001 as compared to SD (Tukey's multiple comparisons post-hoc test)).

FIG. 5E illustrates a graphical depiction showing that genetic loss of PPARγ does not alter blood glucose (n=4-9 mice; two-way ANOVA, treatment: F=57.43, P<0.001; **P<0.01, ***P<0.001 as compared to SD (Tukey's multiple comparisons post-hoc test)).

FIG. 5F illustrates a graphical depiction showing that genetic loss of PPARγ does not alter weight (n=4-9 mice; two-way ANOVA, interaction: F=4.813, P<0.05, treatment: F=90.6, P<0.001; **P<0.01, ***P<0.001 as compared to SD (Tukey's multiple comparisons post-hoc test)) of PPARγ2WT, PPARγ2HET and PPARγ2KO littermates (P30).

FIG. 5G illustrates a graphical depiction showing the effect of KD-treatment on blood β-hydroxybutrate (n=3-9 mice; two-way ANOVA, treatment: F=20.91, P<0.001; *P<0.05, ***P<0.001 as compared to SD (Holm-Sidak's multiple comparisons post-hoc test)).

FIG. 5H illustrates a graphical depiction showing the effect of KD-treatment on blood glucose (n=3-11 mice; two-way ANOVA, treatment: F=27.96, P<0.001; *P<0.05, **P<0.01 as compared to SD (Tukey's multiple comparisons post-hoc test)).

FIG. 5I illustrates a graphical depiction showing the effect of KD-treatment on weight (n=3-11 mice; two-way ANOVA, treatment: F=12.21, P<0.01; **P<0.01 as compared to SD (Tukey's multiple comparisons post-hoc test)) of PPARγ^{fl/fl} -NKO and PPARγ^{fl/fl} mice (P30).

FIG. 5J illustrates a graphical depiction showing that pioglitazone (10 mg/kg/day, i.p., for five days) does not affect blood β-hydroxybutrate concentrations.

FIG. 5K illustrates a graphical depiction showing that pioglitazone (10 mg/kg/day, i.p., for five days) does not affect blood glucose concentrations.

FIG. 5L illustrates a graphical depiction showing that pioglitazone (10 mg/kg/day, i.p., for five days) does not affect weight of wild-type (WT) or Kv1.1 knockout (Kv1.1KO) as compared to vehicle injected mice (P40).

FIG. 6A illustrates a graphical depiction showing that in vivo pioglitazone- and KD-treatment result in similar improvements in in vitro measurements of mitochondria function, synaptic plasticity and network excitability and the effect of genotype, dietary treatments and PIO on oxygen consumption during State III respiration of mitochondrial respiratory complex I (MRCI) of isolated cortical mitochondria (n=3-7 mice; one-way ANOVA, treatment: F=4.486, P<0.05; *P<0.05 as compared to WT (Tukey's multiple comparisons post-hoc test)).

FIG. 6B illustrates a graphical depiction showing that in vivo pioglitazone- and KD-treatment result in similar improvements in in vitro measurements of mitochondria function, synaptic plasticity and network excitability and the effect of hippocampal mossy fiber-CA3 synaptic plasticity (n=3-6; one-way ANOVA, treatment: F_{(3,14)}=8.342, P<0.01; ***P<0.001 as compared to WT (Tukey's multiple comparisons post-hoc test)).

FIG. 6C illustrates a graphical depiction showing that in vivo pioglitazone- and KD-treatment result in similar improvements in in vitro measurements of mitochondria function, synaptic plasticity and network excitability and the incidence of CA3 network high frequency oscillations in the ripple band (100-175 Hz) (n=8 slices; one-way ANOVA, treatment: F=6.082, P<0.01; **P<0.01 as compared to WT (Tukey's multiple comparisons post-hoc test)).

FIG. 6D illustrates a graphical depiction showing that in vivo pioglitazone- and KD-treatment result in similar improvements in in vitro measurements of mitochondria function, synaptic plasticity and network excitability and the incidence of CA3 network high frequency oscillations in the pathologic fast ripple band (200-600 Hz) (n=8 slices; one-way ANOVA, treatment: F=4.601, P<0.05; *P<0.05, **P<0.01 as compared to Kv1.1KO (Tukey's multiple comparisons post-hoc test)).

### DETAILED DESCRIPTION

Overview

Approximately 30% of people with epilepsy do not achieve adequate seizure control with current anti-seizure drugs. The high fat, low carbohydrate ketogenic diet (KD) is a highly effective therapeutic option for this non-responsive population completely abolishing seizures in 7-10% of patients and reducing seizure frequency by >50% in two-thirds of patients. Due to the strict regimen of the KD, it is primarily prescribed to pediatric patients but is also effective in adult epilepsies. Despite compliance issues in adults, the KD is currently under investigation as treatment for other neurodegenerative diseases such as Alzheimer's disease, Parkinson's disease, and Amyotrophic Lateral Sclerosis (ALS). Although the mechanisms of KD anti-seizure efficacy are not completely understood, the KD has been demonstrated to involve disease-modifying pathways including central anti-inflammatory and anti-oxidant pathways and those that improve mitochondrial function.

The KD is thought to engage the peroxisome proliferator activated receptor α (PPARα), a type II nuclear transcription factor, to regulate lipid and energy metabolism. PPARα regulates genes involved in fatty acid catabolism and the production of ketone bodies (β-hydroxybutyrate, acetoacetate and acetone), which replace glucose as a deliverable fuel source to other tissues, including the brain. PPARα agonists have been shown to decrease neuroinflammation, but it is the PPARγ isoform that regulates anti-inflammatory, anti-oxidant and mitochondrial genes similar to the KD. Agonists for either PPARα or PPARγ isoforms provide beneficial effects in a variety of neurodegenerative disorders, including protection against acute chemoconvulsant-induced seizures.

Accordingly, a composition, a method for administering an anti-seizure composition, and a method for treating a seizure are described. In an implementation, a composition comprises a peroxisome proliferator activated receptor gamma (PPARγ) antagonist that is administered to an individual, where the individual has been administered a ketogenic diet. In an implementation, a method for administering an anti-seizure composition comprises administering a ketogenic diet to an individual; and concurrently administering a therapeutically effective dose of a peroxisome proliferator activated receptor gamma (PPARγ) antagonist to the individual In an implementation, a method for treating a seizure comprises administering a therapeutically effective dose of a peroxisome proliferator activated receptor gamma (PPARγ) antagonist to an individual on a ketogenic diet and suffering from seizures.

Example Implementation

In the examples described herein, the nutritionally-regulated transcription factor peroxisome proliferator activated receptor gamma, PPARγ, contributes to the anti-seizure efficacy of the KD. Spontaneous recurrent seizures were quantified in epileptic Kv1.1 knockout (KO) mice with 24 hr video-EEG. Expression of nuclear PPARα, PPARγ1 and PPARγ2 protein in brain homogenates was determined with western blots. Acute seizures were provoked in control, PPARγ2KO, and neuron-specific PPARγKO (NKO) mice with flurothyl vapors. Respiration of isolated hippocampal mitochondria was determined with oxygen polarography. Acutely isolated hippocampal slices were used for in vitro electrophysiology.

The KD increased nuclear PPARγ2 in WT and Kv1.1KO mice, but did not affect PPARα or PPARγ1. A PPARγ antagonist prevented KD-mediated changes in PPARγ2 expression and prevented the anti-seizure efficacy of the KD. KD prolonged the latency to flurothyl-induced seizure in WT mice, but this effect was lost in PPARγ2KO mice and NKO mice. PPARγ agonism mimicked KD effects on PPARγ2 expression, reduced seizures, dampened network hyperexcitability and restored hippocampal mitochondria function in epileptic Kv1.1KO mice. Modulation of PPARγ had no effect on blood β-hydroxybutyrate or glucose regardless of whether or not the animal consumed a KD.

One specific study testing the efficacy of administering a ketogenic diet in conjunction with administering a peroxisome proliferator activated receptor gamma (PPARγ) antagonist for reducing, preventing, and/or eliminating seizures, including epileptic seizures and refractory seizures, is described below and illustrated in the figures. The study described below shows that PPARγ2 is a therapeutic target for refractory epilepsy and that the ketogenic diet combined with a PPARγ antagonist is effective for treating and/or preventing seizures.

Animals. The Mice were housed in a temperature (e.g., 25° C.) and humidity (e.g., 50-60%) controlled and pathogen-free environment. The mice were given food and water ad libitum and kept on a 12-hour light/dark cycle. Heterozygous Kcnal-null mice on a C3HeB/FeJ congenic background were purchased from Jackson Laboratories (Bar Harbor, Maine) and bred to obtain Kv1.1 wild-type (WT) and Kv1.1KO littermates. Heterozygous Pparγ2-null mice on a mixed 129sv-C57B⅙ background were provided by Gema Medina-Gomez (Universidad Rey Carlos, Madrid, Spain) and bred to obtain PPARγ2WT, PPARγ2Het and PPARγ2KO littermates. Tail clips were taken at postnatal day (P)10-P15 and sent to Transnetyx Inc. for genotyping (Cordova, Tenn., U.S.A.). Homozygous foxed Pparγ^{fl/fl} and homozygous neuronal-specific synapsin I-Cre⁺Pparγ^{fl/fl} knockout (Pparγ^{fl/fl}-NKO; Cre expression driven by synapsin I) mice were provided by J. M. Olefsky (University of California—San Diego) and M. W. Schwartz (University of Washington) and bred to provide control Pparγ_{fl/fl} mice and Pparγ^{fl/fl}-NKO mice. All procedures involving animals were in accordance with National Institutes of Health guidelines, the EU Directive 2010/63/EU and were approved by the Institutional Animal Care and Use Committees at Creighton University School of Medicine.

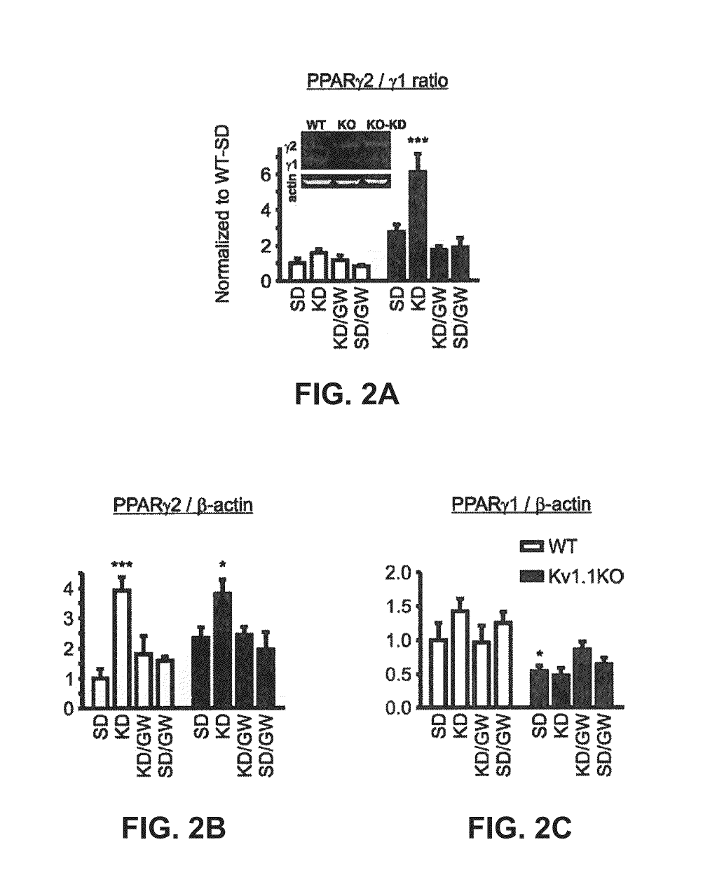

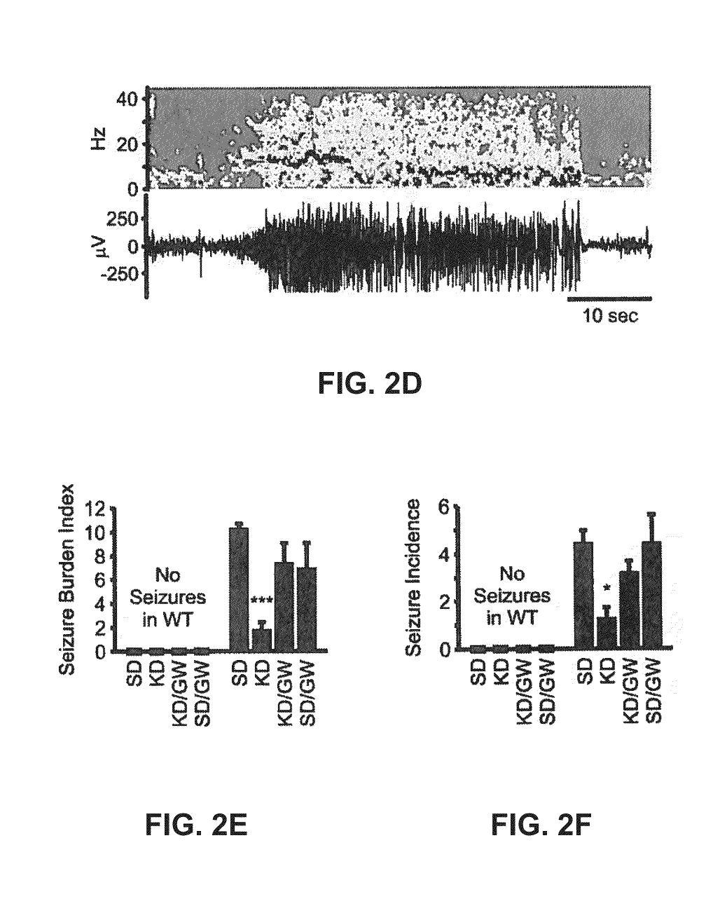

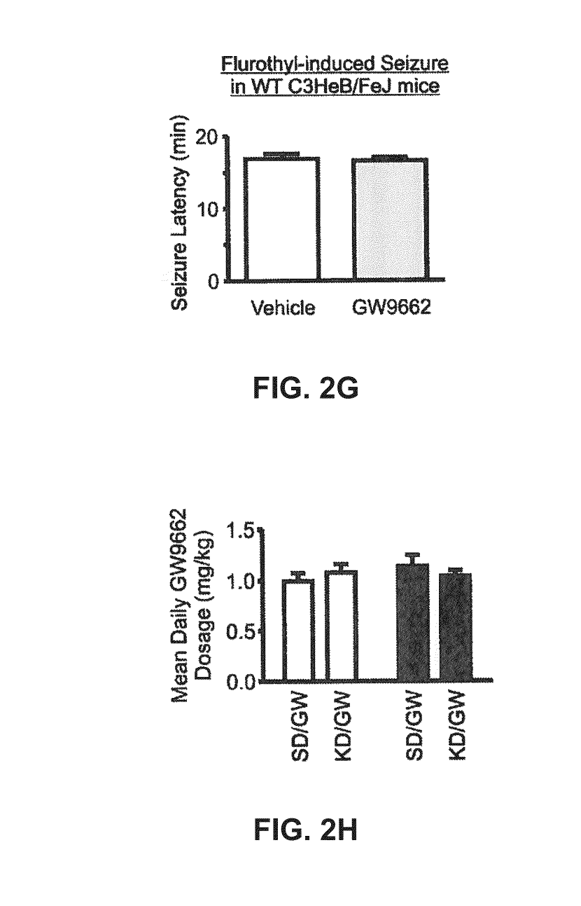

Dietary and pharmacological treatments. On P21, mice were randomly weaned onto either a standard diet (SD) or a KD (6.3:1, fat to carbohydrates plus proteins; Bio-Sery F3666, Frenchtown, N.J., U.S.A.) for 10-14 days. For dietary experiments involving Kv1.1WT and Kv1.1KO mice, drinking water contained either 0.0323% DMSO vehicle or GW9662 (2.68 mg/ml) to obtain an average dosage of 1.062±0.038 mg/kg/day (n=18). There was no difference in the average daily water consumption or resulting GW9662 dosage between experimental groups (see FIG. 2H). For the pioglitazone experiments, on P32-33 Kv1.1KO mice were intraperitoneal-injected with saline vehicle for two days, followed by six days of daily pioglitazone injections (10 mg/kg/day, i.p., at 9:00AM). Three hours after the last pioglitazone injection, mice were euthanized and brains processed for western blot, electrophysiology, or mitochondria experiments.

Blood β-hydroxybutyrate and glucose measurements. β-hydroxybutyrate and glucose levels were measured every three days from blood samples collected from the tail vein of each mouse using a test strip system and reader (Precision Xtra Advance Diabetes Management System with Precision Xtra blood ketone test strips and blood glucose test strips; Abbott Diabetes Care Inc., Alameda, Calif., U.S.A.).

Intracranial electroencephalography electrode implantation and seizure analysis. On P27, mice underwent surgical implantation of electrodes. Mice were maintained under isoflurane anesthesia and normothermic conditions. One ground (1.5 mm anterior lamba, 1.5 mm lateral) and two subdural (1 mm posterior Bregma, 1.5 mm bilateral) intracranial cortical electroencephalographic (iEEG) electrodes were implanted, secured, and attached to a headmount. Following a 5-6 day recovery, seizures were monitored for forty-eight hours using a continuous infrared video surveillance system time-synced to an EEG recording system (Pinnacle Technology, Inc., Lawrence, Kans., U.S.A). EEG recordings were acquired with a 250 Hz sampling rate and band-pass filtered between 0.5 and 40 Hz. During seizure monitoring, mice were single-housed in hexagon cages. EEG recordings were imported into Spike2 v6-7 software (Cambridge Electronic Design, Cambridge, England) for initial seizure identification using time-frequency analysis. Subsequently, EEG seizures were confirmed using Sirenia software (Pinnacle Technology, Inc.) that time-synced EEG and video recordings and behavioral manifestations were manually verified by 2 blinded investigators. Incidence and severity of each seizure was scored for the first 15 minutes of each hour during the time period of highest seizure occurrence for Kv1.1KO mice (00:00-08:00). Seizure severity was scored using a modified Racine scale: 0—normal; 1—myoclonic jerk or “pop;” 2—side-to-side head movement; 3—forelimb/hindlimb clonus, tail extension, a single rearing event; 4—continuous rearing and falling; 5—severe tonic-clonic seizures. To weigh the incidence and severity of seizures, seizure burden index (SBI) scores were calculated using the equation: SBI=[Σ(σiγi)]/ε, where σ indicates the severity; i indicates each stage of seizure (1-5); γ is the incidence; and ε indicates the total number of epochs scored.

Immunofluorescent western blot. Nuclei were isolated from whole brain homogenate using a nuclear extraction kit according to manufacturer instructions (AY2002; Affymetrix, Inc., Santa Clara, Calif., U.S.A.). Nuclear extracts were mixed with Laemmli's loading buffer (Bio-Rad, Hercules, Calif., U.S.A.) containing beta-mercaptoethanol (Sigma-Aldrich, St. Louis Mo., U.S.A.), heated to 99° C. and run through precast PAGE gels (Bio-Rad). Proteins were transferred to Immobilon-FL PVDF membranes (EMD Millipore, Billerica, Mass., U.S.A.), which were then blocked in Odyssey blocking buffer (OBB; Li-Cor Biosciences, Lincoln, Nebr., U.S.A.) and phosphate buffered saline (PBS) at a 1:1 ratio for one hour at room temperature (RT). Membranes were incubated with the following primaries overnight at 4° C.: rabbit anti-PPARγ (1:400; 07-466, EMD Millipore; recognizes PPARγ1 at 52 kDa and PPARγ2 at 56 kDa) or mouse anti-PPARα (1:500; MAB3890, EMD Millipore). (β-actin was detected using the following antibodies: rabbit anti-β-actin (1:10,000; 04-1116, EMD Millipore) or mouse anti-β-actin (1:8,000; 926-42212, Li-Cor Biosciences). After PBS-T washes, membranes were incubated in secondary antibodies for one hour at RT: goat anti-rabbit (1:5,000; 926-32221, Li-Cor Biosciences) or goat anti-mouse (1:20,000-1:40,000; 926-32210, Li-Cor Biosciences). Membranes were washed in PBS-T and rinsed in distilled water. Samples were run in duplicate on each gel. Densitometric analysis was conducted using images captured on an Odyssey FC (Licor, Lincoln, Nebr.), and PPAR protein signal was normalized to within well β-actin values. Averages of the duplicates were determined and values normalized to the WT-standard diet values within each gel.

Flurothyl-induced Seizures. All experiments were performed in a fume hood. Mice were acclimated 1 hour before testing. Mice were individually placed in a 2.7 L airtight glass chamber. A 10% solution (in 95% ethanol) of flurothyl (bis-2,2,2-trifluoroethyl ether; Sigma-Aldrich, St. Louis, Mo., U.S.A.) was delivered by a syringe pump (KD Scientific, Holliston, Mass., U.S.A.) at a constant rate of 0.05 ml/min and allowed to drip onto a Whatman grade 1 filter paper until the mouse reached a generalized tonic-clonic seizure with loss of posture. Seizure latency was measured from the first drip of flurothyl onto the filter paper to the onset of the generalized tonic-clonic seizure.

Acute hippocampal slice preparation and multielectrode array recordings. Mice (P33-42) were anesthetized with isoflurane, decapitated, and their brains removed and quickly placed into ice cold, oxygenated (95% O₂/5% CO₂) artificial cerebrospinal fluid (aCSF) containing (in mM): 206 Sucrose, 2.8 KCl, 1 CaCl₂, 1 MgCl₂, 2 MgSO₄, 26 NaHCO₃, 1.25 NaH₂PO₄, and 10 glucose (pH 7.4). Horizontal sections (350 μM) of ventral hippocampal-entorhinal cortex (HEC) were cut on a Leica VT1200 (Leica Microsystems Inc., Bannockburn, Ill., USA) and transferred to a holding chamber for 1 hr with warm (32° C.) oxygenated aCSF containing (in mM): 125 NaCl, 3.0 KCl, 2.4 CaCl₂, 2.5 MgSO₄, 26 NaHCO₃, 1.25 NaH₂PO₄, and 10 glucose (pH=7.4). A custom probe cap allowed delivery of humidified air (95% O₂/5% CO₂) and perfusion (1 ml min⁻¹) of in-line pre-warmed oxygenated aCSF that resulted in a solution inlet temperature of ˜33.2° C. and an outlet temperature of ˜31.6° C. The bath level was maintained near interface. Spontaneous and evoked extracellular potentials were recorded with Mobius v2 software (Witwerx Inc., Tustin, Calif., USA) and acquired at a 20 kHz sampling rate with a bandwidth of 0.1 Hz-10 kHz. Post-synaptic field potentials and short-term plasticity of the mossy fiber (MF)-CA3 synapse were obtained by successive paired stimulations (50 ms inter-stimulus interval, 50% maximal intensity) of Dentate granule cell mossy fibers located in the hilar region and recorded in CA3 stratum lucidum. Paired pulse facilitation was calculated by dividing the slope (10-90%) of stimulation 2 by the slope of stimulation 1 and presented as the percent above stimulation 1. For analyses of spontaneous local field potentials, data was imported into Spike2 (v7) software (Cambridge Electronic Design, Ltd., Cambridge, England) as previously described. The incidence of sharp waves with nested ripples and/or fast ripples was quantified using time-frequency representations of raw recordings generated with short-time Fourier transform (STFT) algorithms in Spike2 software. STFTs were performed with mean detrending, Hanning windowing, and FFT size of 2048 points.

Mitochondrial isolation and oxytherm polarography. Mice were anesthetized with isoflurane and cortical tissue was quickly micro-dissected on ice. Tissue was homogenized in isolation buffer (5×V/V in mM: 215 mannitol, 75 sucrose, 1 EGTA 20 HEPES and 0.1% BSA, pH 7.2 adjusted with KOH) and mitochondria were isolated using differential centrifugation. Isolated mitochondria were resuspended in isolation buffer without EGTA. Protein concentrations were determined with Bradford assay. Mitochondria (100 μg/mL) were resuspended in KCl respiration buffer (in mM: 125 KCl, 20 HEPES, 2 MgCl₂, 2.5 KH₂PO₄, pH 7.2) and placed in a sealed, thermostatically controlled chamber at 37° C. Oxygen polarography was measured using a standard Clark-type electrode (Hanstech Instruments, Ltd., Norfolk, England). The following parameters were measured sequentially in duplicates per sample: Mitochondria were energized via ATP-producing NADH:coenzyme Q oxidoreductase (complex I)-driven state III respiration with 5 mM pyruvate and 2.5 mM malate (PM) and concurrent activation of ATP synthase with 150 μM adenosine diphosphate; state IV respiration was initiated with oligomycin-induced inhibition of ATP synthase (1 μM); maximal respiratory rate (state V) was measured in the presence of the chemical protonophore p-triflourometh-oxyphenylhydrazone (FCCP; 1 μM); and MRCI-respiratory dependency was verified in the presence of the MRCI inhibitor rotenone. Only samples with a respiratory control ratio greater than 4 were analyzed. Unbiased assessment of the rate of oxygen consumption was determined using computer-generated line of best-fit algorithm and nmol of oxygen mg⁻¹ min⁻¹ was calculated.

Reagents and Statistics. Unless otherwise specified, all reagents were purchased from Sigma-Aldrich. Statistical significance was determined with Prism6 software (Graphpad Software Inc., La Jolla, Calif., U.S.A.) using two-way ANOVA for genotype and treatment with an appropriate post hoc test unless otherwise stated.

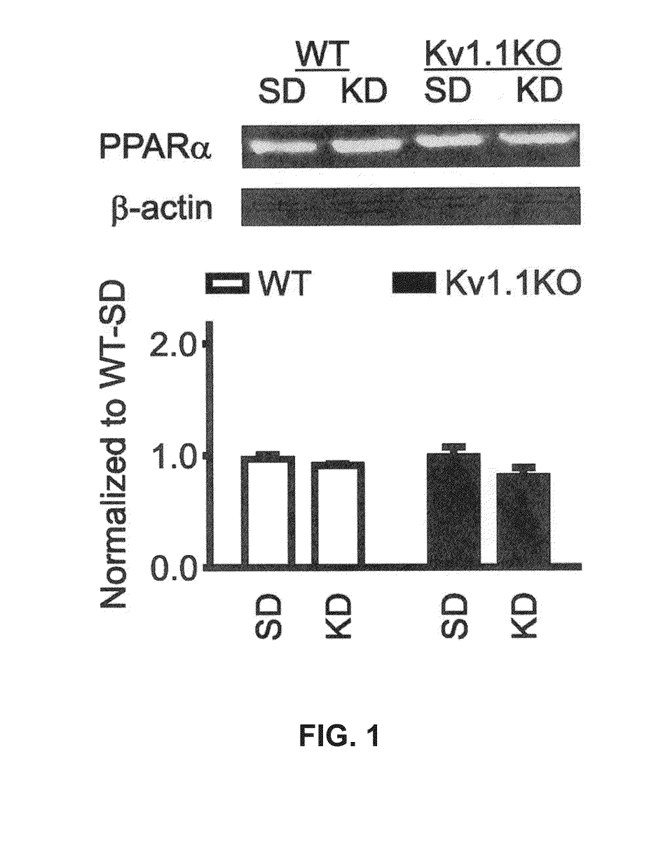

The Kv1.1KO mouse is a model of developmental epilepsy and sudden unexpected death in epilepsy (SUDEP) that exhibits severe, spontaneous seizure phenotypes. The KD has been shown to be highly efficacious in attenuating the spontaneous recurrent seizures in Kv1.1KO mice. We examined KD modulation of nuclear expression of PPARα and the two splice variants of PPARγ (γ, γ2) in homogenates of mouse brain tissue from wild-type (WT) and Kv1.1 knockout (Kv1.1KO) littermates. Brain PPARα was unaffected by genotype or KD-treatment (see FIG. 1). In contrast, the two PPARγ splice variants were differentially regulated between genotypes with PPARγ1 predominating in WT brain and PPARγ2 predominating in Kv1.1KO brain, resulting in a significantly larger PPARγ2/γ1 ratio (see FIGS. 2A-C). Seizures were quantified for incidence and further weighted for Racine scale severity to calculate a seizure burden index score for each mouse. A KD treatment decreased seizures by ˜75% and distinctively increased PPARγ2 in both genotypes, which further increased the Kv1.1KO PPARγ2/γ1 ratio (see FIGS. 2A-F).

To determine whether KD regulation of nuclear PPARγ2/γ1 ratio and seizures was due to PPARγ activation, the PPARγ antagonist, GW9662 (˜1 mg/kg/day) (see FIG. 2H), was co-administered (or “concurrently administered”) throughout KD treatment. GW9662 prevented KD-mediated changes in nuclear PPARγ2 and anti-seizure efficacy (see FIGS. 2A-F). Importantly, GW9662 did not worsen Kv1.1KO seizures, induce spontaneous seizures in WT mice, nor did it reduce seizure thresholds of WT mice exposed to flurothyl (see FIGS. 2E-G). These data indicate that the antagonist does not unmask a convulsant mechanism and support that PPARγ is directly involved in the anti-seizure mechanisms of the KD.

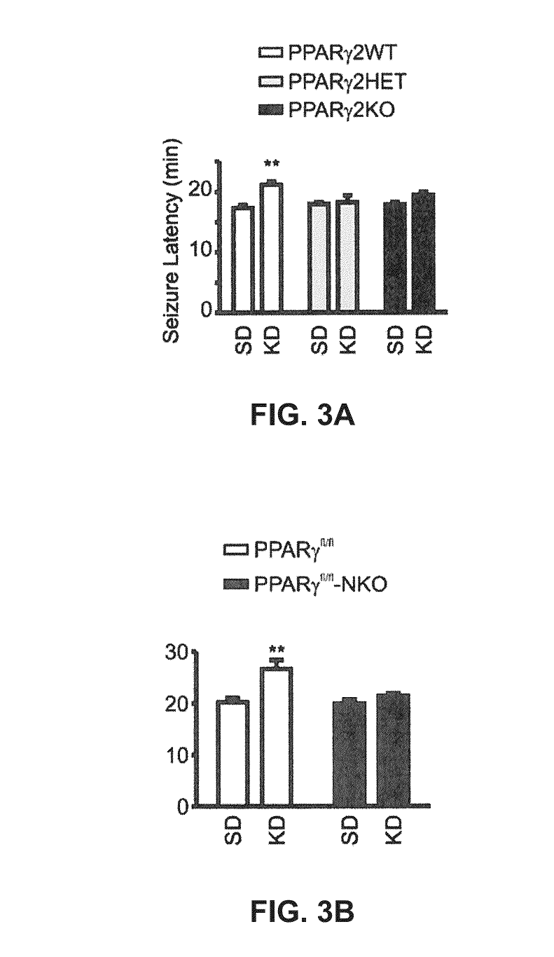

The necessity of PPARγ2 in KD anti-seizure efficacy using mice lacking PPARγ2 and mice lacking PPARγ specifically in neurons was further determined. PPARγ2 knockout (PPARγ2KO) mice, WT, and heterozygous littermates were fed either a standard diet or KD for two weeks, at the end of which the latency to flurothyl-induced generalized tonic-clonic seizures was measured. KD-treatment increased seizure latency of WT mice, but failed to protect heterozygous and PPARγ2KO littermates (see FIG. 3A). We also found that neuronal PPARγ is necessary for KD-mediated seizure protection. KD-treatment was ineffective in neuron-specific Synapsin I-Cre⁺ PPARγ^{fl/fl} knockout mice, whereas seizure latencies of PPARγ^{fl/fl} control mice fed a KD were significantly increased (see FIG. 3B).

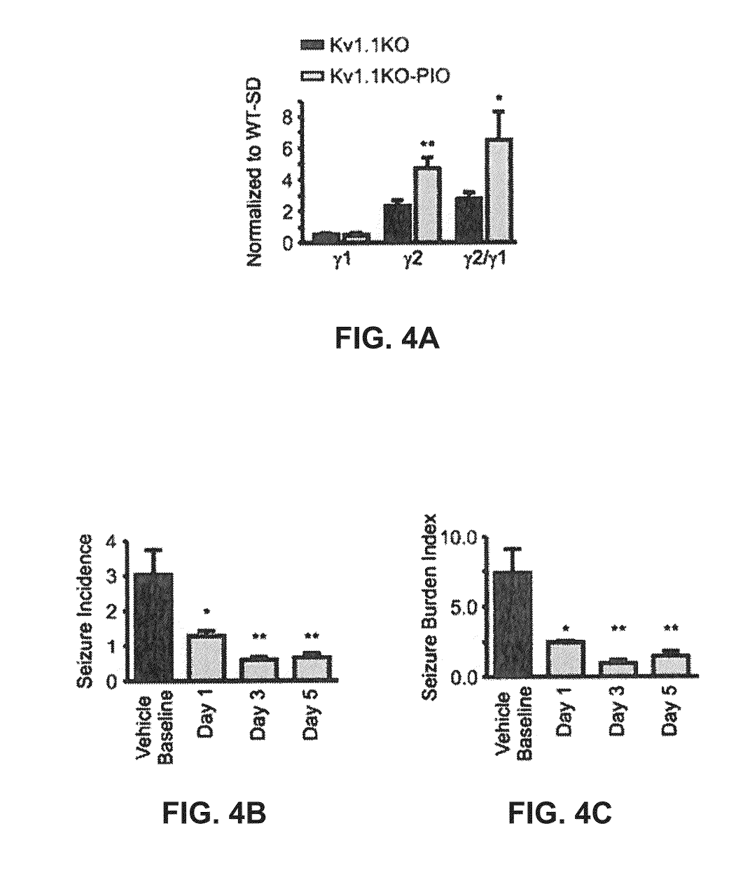

Whether pioglitazone, a PPARγ agonist and Type II Diabetes Mellitus therapeutic thiazolidinedione compound, mimicked effects similar to the KD in Kv1.1KO mice was also determined. Kv1.1KO mice were treated with a saline vehicle (i.p. injection) for two days followed by five days of pioglitazone treatment (10 mg/kg/day, i.p.). Similar to KD, pioglitazone increased nuclear PPARγ2 in Kv1.1KO brain homogenates and decreased seizure incidence burden over a five-day treatment (see FIGS. 4A-C). These results support that PPARγ activation is sufficient to reduce seizures in an animal with a severe seizure phenotype.

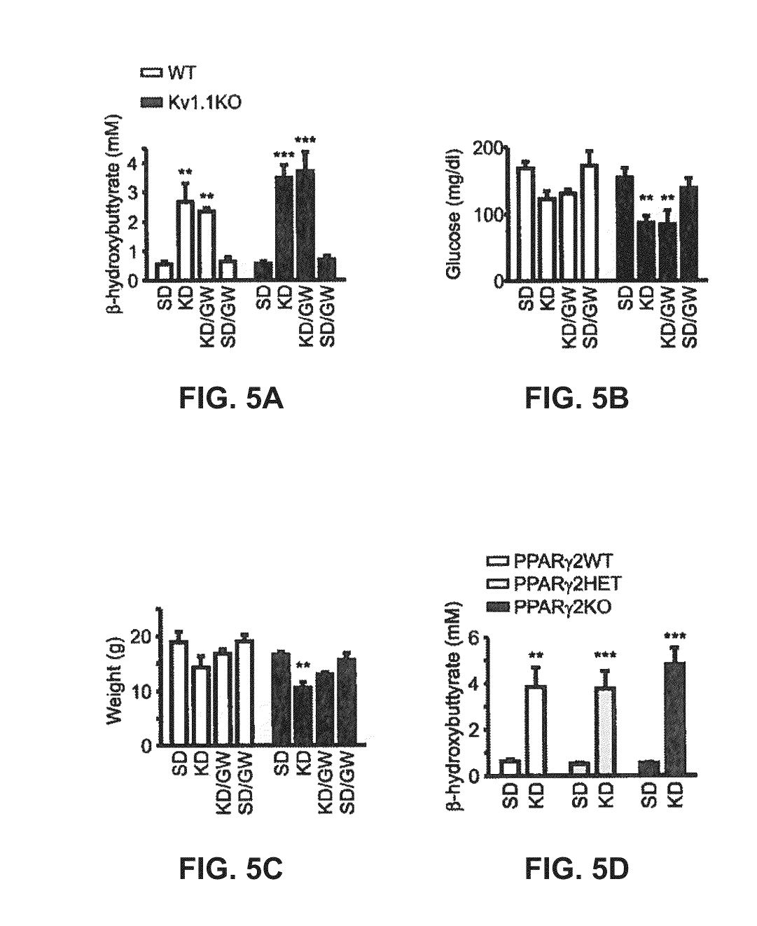

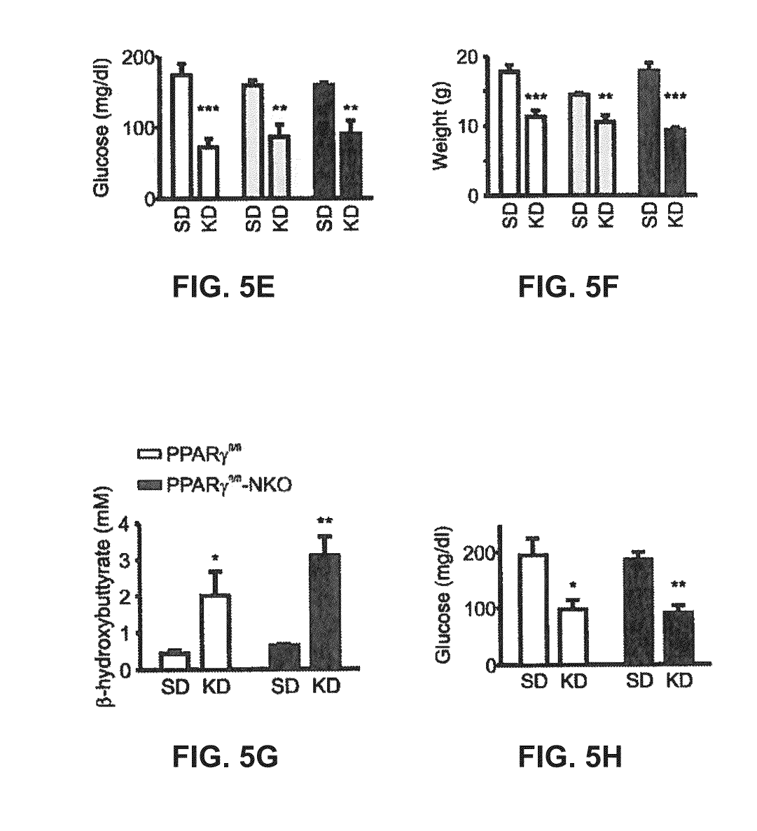

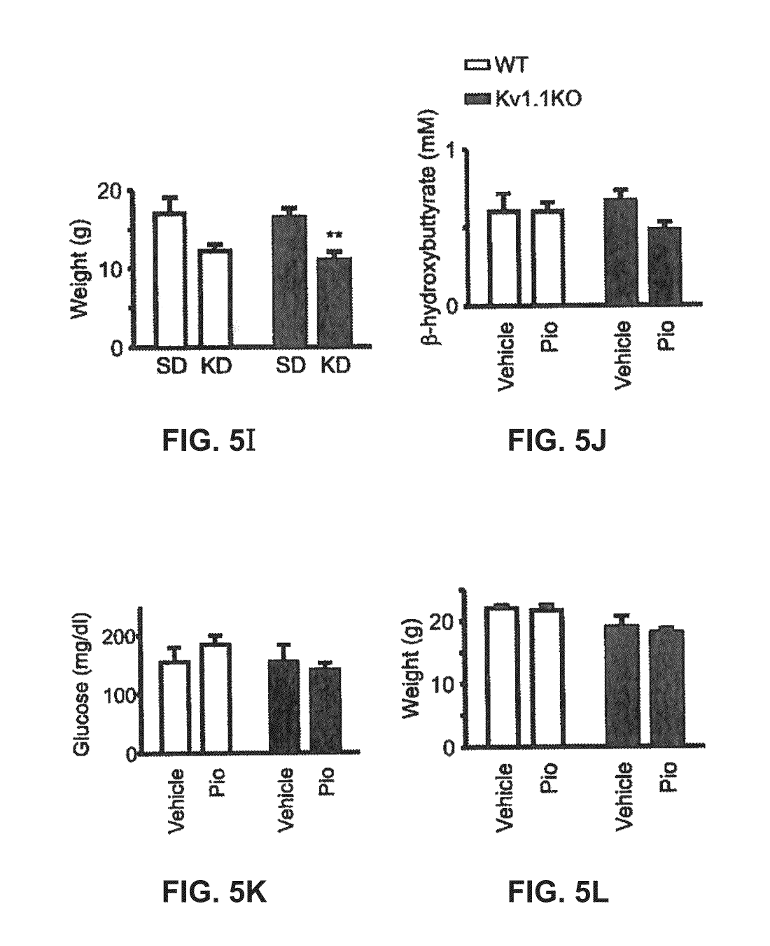

Peripheral increases in blood ketone bodies (e.g., β-hydroxybutyrate) and/or reductions in blood glucose are hypothesized to be critical components of the mechanism of the KD. To rule out the possibility that our pharmacologic or genetic manipulations of PPARγ may have had indirect central effects by attenuating KD effects on peripheral ketone bodies and glucose, we measured blood concentrations of β-hydroxybutyrate and glucose of mice in all of our experimental groups. We found that antagonizing PPARγ with GW9662 did not affect KD modulation of concentrations of blood β-hydroxybutyrate, blood glucose, or body weight (see FIG. 5A-C). Similarly, genetic loss of PPARγ2 or neuronal PPARγ had no effect on these parameters (see FIG. 5D-I). Also, the PPARγ agonist pioglitazone did not change blood β-hydroxybutyrate, glucose, or body weight in either Kv1.1WT or Kv1.1KO mice (see FIG. 5J-L). These results suggest that peripheral and central PPARγ is not involved in regulating blood ketone bodies or glucose in normal and epileptic mice fed a standard diet or KD and that central PPARγ is directly involved in an inducible anti-seizure mechanism in these mouse models.

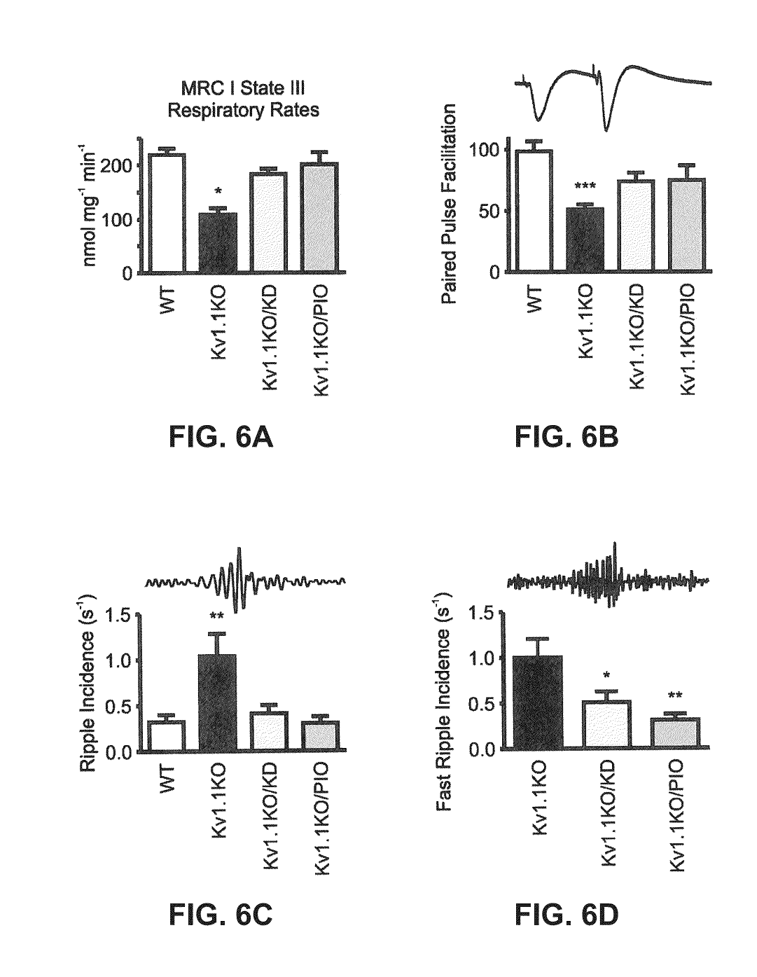

Whether in vivo treatment with pioglitazone and KD exerted similar effects on in vitro measurements of brain mitochondria function, synaptic transmission and network excitability was determined. Previously, it was determined that Kv1.1KO hippocampal glutamatergic mossy fibers have decreased paired pulse facilitation inversely indicating increased neurotransmitter release probabilities, which contribute to increased generation of high frequency oscillations (HFOs) with emergent pathological fast ripples in the CA3 region. In subsequent studies, it was found that in vivo KD treatment ameliorated these synaptic and network pathophysiologies. Further, it was found that reactive oxygen species (ROS)-mediated impairment of mitochondrial complex I respiration contribute to these pathophysiologies and seizure phenotype. Here, it was found that in vivo treatment with pioglitazone and KD had similar effects; both improving mitochondrial respiration by ˜80%, increasing mossy fiber paired pulse facilitation by ˜45% and attenuating HFO incidence by ˜60% (see FIGS. 6A-D). Together, these results show that PPARγ2 activation attenuates the pathophysiology of Kv1.1KO mice on several functional levels (e.g., synaptic, network, mitochondrial), which may contribute to reduce seizure-genesis.

There are four principal findings of the study described herein. First, the KD increases nuclear expression of PPARγ2 in the brain, but does not affect the expression of PPARγ. Second, pharmacologic and genetic loss of PPARγ abolishes KD anti-seizure protection against acute flurothyl-induced seizures in non-epileptic mice and chronic spontaneous recurrent seizures in epileptic mice. Third, exogenous administration of a PPARγ agonist mimics KD effects on nuclear PPARγ2 expression in the brain, reduces seizures, dampens network hyperexcitability, and restores hippocampal mitochondria function in epileptic mice. Fourth, modulation of PPARγ has no effect on blood β-hydroxybutyrate or glucose regardless of whether the animal consumes a standard diet or KD. Collectively, the data implicate brain PPARγ2 among the mechanisms by which the KD reduces seizures.

The KD and PPARγ agonists regulate similar anti-inflammatory, anti-oxidant and pro-mitochondrial pathways. These include, but are not limited to, upregulation of IκB, inhibition of NFκB, reduction of cytokines such as IL-1β, IL-6 and TNF-α, upregulation of genes encoding mitochondrial enzymes involved in oxidative phosphorylation (e.g., multiple subunits of complexes I, II, IV and V), induction of mitochondrial biogenesis and upregulation of uncoupling protein 2, catalase and glutathione. Previous studies found that mitochondria isolated from cortical and hippocampal tissue from epileptic Kv1.1KO mice had reduced ATP producing mitochondrial respiratory complex I (MRCI)-driven respiration which appeared to be due to post-translational inhibition by elevated reactive oxygen species (ROS). Associated with this mitochondrial dysfunction was a decrease in paired pulse facilitation at the Kv1.1KO mossy fiber-CA3 synapse indicating increased neurotransmitter release. This synaptic phenotype was mimicked by acute application of the MRCI inhibitor rotenone to WT hippocampal slices and resulted in cellular and network hyperexcitability and exacerbated provoked seizure-like events in vitro. The current experiments demonstrate that treating an epileptic Kv1.1KO mouse with either the KD or pioglitazone rescues MRCI respiration, restores paired pulse facilitation and dampens network hyperexcitability. One anti-seizure mechanism of KD modulation of PPARγ is reducing ROS and restoring mitochondrial function. This would result in increased mitochondrial ATP production and cytosolic calcium buffering capacity. Several studies have demonstrated that mitochondria regulate synaptic transmission via their production of ATP and reactive oxygen species (ROS) and their sequestration of cytosolic calcium. Under these circumstances, extracellular adenosine and glutamate would increase and decrease, respectively, leading to additional dampening of excitability, decreased excitotoxicity, and decreased inflammation, which have been proposed to participate in anti-seizure and neuroprotective mechanisms of the KD.

Endogenous agonists of PPARγ include unsaturated fatty acids, eicosanoids, oxidized lipids and nitroalkenes. All of these natural ligands increase during seizures and may cause the increase in nuclear PPARγ2 observed in Kv1.1KO mice fed a standard diet; thus, illustrating an endogenous protective mechanism induced by the disease itself. As to the selective increase of PPARγ2 over PPARγ1, this may be due to an additional 30 amino acids that convey a 5-10 fold more effective ligand-independent activation to PPARγ2 relative to PPARγ1. Also, PPARγ2 is the only PPARγ isoform regulated at the transcriptional level by nutrition. Transcriptional co-factors and post-translational modifications are also likely to result in tissue-specific differences in the regulation of gene sets by the PPARγ splice variants. This is evident in the periphery where PPARγ2, but not PPARγ, is induced during high fat diets and initiates adipogenesis, increases lipid-buffering and reduces lipotoxicity.

The lack of effect of PPARγ agonists on blood glucose and blood β-hydroxybutyrate regardless of genotype or dietary treatment and the loss of KD protection in neuronal PPARγ knockout mice support a direct anti-seizure effect of central PPARγ. Many studies have demonstrated the anti-seizure effects of low carbohydrates and ketone bodies directly or during KD or calorie restriction. These factors may be upstream of PPARγ; however, the degree and direction of PPARγ modulation appears to be tissue dependent, age-dependent, species-dependent and duration of treatment-dependent.

The results of the study show that central PPARγ2 is a therapeutic target for refractory epilepsy. Further, the findings and the overlap of studies investigating the efficacy of the KD and PPARγ agonists in stroke, Alzheimer's disease, Parkinson's disease and ALS suggest that KD effects in these neurodegenerative diseases may also be via PPARγ. It is possible that modulation of PPARγ2 may provide a common pathway linking the multitude of hypotheses surrounding the KD.

Exemplary composition

In an implementation, one composition that may be used as an anti-seizure composition, includes a peroxisome proliferator activated receptor gamma (PPARγ) antagonist that is administered to an individual, where the individual has been administered a ketogenic diet. In some specific embodiments, the PPARγ can include, for example, GW9662 and/or pioglitazone. Additionally, the composition may include different forms, such as a solid, a powder, a liquid, and/or a suspension, which composition may be administered in a variety of ways (e.g., orally, intravenously, exogenously, etc.). Furthermore, the PPARγ antagonist may include a PPARγ2 antagonist.

Exemplary methods

In an implementation, a method for administering an anti-seizure composition includes administering a ketogenic diet to an individual and concurrently administering a therapeutically effective dose of a peroxisome proliferator activated receptor gamma (PPARγ) antagonist to the individual. As described above, the PPARγ antagonist can include GW9662 and/or pioglitazone and can be administered orally, intravenously, and/or exogenously. Administering the anti-seizure composition reduces seizure frequency.

In another implementation, a method for treating a seizure includes administering a therapeutically effective dose of a peroxisome proliferator activated receptor gamma (PPARγ) antagonist to an individual on a ketogenic diet and suffering from seizures. In these implementations, the administering step may be performed by a controller, which can include a processor, memory, and/or a communications interface. The controller can control hardware (e.g., a pump, a valve, etc.) used for the administering step. Additionally, the controller can present information used in the administering step (e.g., dosage information, nutrition information, diet schedules, etc.).

The controller may operate hardware, including some or all components, under computer control. For example, a processor can be included with or in an administration device, controller to control the components and functions of the administration device described herein using software, firmware, hardware (e.g., fixed logic circuitry), manual processing, or a combination thereof. The terms “controller,” “functionality,” “service,” and “logic” as used herein generally represent software, firmware, hardware, or a combination of software, firmware, or hardware in conjunction with controlling the administration device. In the case of a software implementation, the module, functionality, or logic represents program code that performs specified tasks when executed on a processor (e.g., central processing unit (CPU) or CPUs). The program code can be stored in one or more computer-readable memory devices (e.g., internal memory and/or one or more tangible media), and so on. The structures, functions, approaches, and techniques described herein can be implemented on a variety of commercial computing platforms having a variety of processors.

The controller can include a processor, a memory, and a communications interface. In some embodiments, the controller may be integrated into an integrated circuit (IC) with a user interface (e.g., controls, a readout, etc.) for the administration device. In another embodiment, the controller, processor, memory, communications interface, and/or user interface may be integrated into one system-in-package/module and/or one or more could be separate discrete components in an end system (e.g., the administration device, such as a pump, valve, etc.).

The processor provides processing functionality for the administration device and/or controller and can include any number of processors, micro-controllers, or other processing systems, and resident or external memory for storing data and other information accessed or generated by the administration device and/or controller. The processor can execute one or more software programs that implement techniques described herein. The processor is not limited by the materials from which it is formed or the processing mechanisms employed therein and, as such, can be implemented via semiconductor(s) and/or transistors (e.g., using electronic integrated circuit (IC) components), and so forth.

The controller may include a memory. The memory can be an example of tangible, computer-readable storage medium that provides storage functionality to store various data associated with operation of the administration device and/or controller, such as software programs and/or code segments, or other data to instruct the processor, and possibly other components of the administration device and/or controller, to perform the functionality described herein. Thus, the memory can store data, such as a program of instructions for operating the administration device (including its components), and so forth. It should be noted that while a single memory is described, a wide variety of types and combinations of memory (e.g., tangible, non-transitory memory) can be employed. The memory can be integral with the processor, can comprise stand-alone memory, or can be a combination of both. In specific instances, the memory may include a buffer (e.g., a region of a physical memory storage used to temporarily store data while it is being moved from one place to another) and/or datalog for storing sensor data.

The memory can include, but is not necessarily limited to removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In implementations, the administration device, the remotely-powered medical sensor 110, and/or the memory can include removable integrated circuit card (ICC) memory, such as memory provided by a subscriber identity module (SIM) card, a universal subscriber identity module (USIM) card, a universal integrated circuit card (UICC), and so on.

The controller may include a communications interface. The communications interface can be operatively configured to communicate with components of the administration device. For example, the communications interface can be configured to transmit data for storage in the administration device, retrieve data from storage in the administration device, and so forth. The communications interface can also be communicatively coupled with the processor to facilitate data transfer between components of the administration device and the processor (e.g., for communicating inputs to the processor received from a device communicatively coupled with the administration device, and/or controller). It should be noted that while the communications interface is described as a component of an administration device and/or controller, one or more components of the communications interface can be implemented as external components communicatively coupled to the administration device via a wired and/or wireless connection. The administration device can also include and/or connect to one or more input/output (I/O) devices (e.g., via the communications interface), including, but not necessarily limited to a display, a mouse, a touchpad, a keyboard, and so on.

The communications interface and/or the processor can be configured to communicate with a variety of different networks, including, but not necessarily limited to: a wide-area cellular telephone network, such as a 3G cellular network, a 4G cellular network, or a global system for mobile communications (GSM) network; a wireless computer communications network, such as a WiFi network (e.g., a wireless local area network (WLAN) operated using IEEE 802.11 network standards); an internet; the Internet; a wide area network (WAN); a local area network (LAN); a personal area network (PAN) (e.g., a wireless personal area network (WPAN) operated using IEEE 802.15 network standards); a public telephone network; an extranet; an intranet; and so on. However, this list is provided by way of example only and is not meant to limit the present disclosure. Further, the communications interface can be configured to communicate with a single network or multiple networks across different access points.

Generally, any of the functions described herein can be implemented using hardware (e.g., fixed logic circuitry such as integrated circuits), software, firmware, manual processing, or a combination thereof. Thus, the blocks discussed in this disclosure generally represent hardware (e.g., fixed logic circuitry such as integrated circuits), software, firmware, or a combination thereof. In the instance of a hardware configuration, the various blocks discussed in the above disclosure may be implemented as integrated circuits along with other functionality. Such integrated circuits may include all of the functions of a given block, system, or circuit, or a portion of the functions of the block, system, or circuit. Further, elements of the blocks, systems, or circuits may be implemented across multiple integrated circuits. Such integrated circuits may comprise various integrated circuits, including, but not necessarily limited to: a monolithic integrated circuit, a flip chip integrated circuit, a multichip module integrated circuit, and/or a mixed signal integrated circuit. In the instance of a software implementation, the various blocks discussed in the above disclosure represent executable instructions (e.g., program code) that perform specified tasks when executed on a processor. These executable instructions can be stored in one or more tangible computer readable media. In some such instances, the entire system, block, or circuit may be implemented using its software or firmware equivalent. In other instances, one part of a given system, block, or circuit may be implemented in software or firmware, while other parts are implemented in hardware. In a specific embodiment, an authentication function or other parts and functions of the system of the remotely-powered medical sensor 110 and/or administration device can be implemented on a remote system (e.g., a server). In this specific embodiment, the administration device may function as a data conduit.

It is to be understood that embodiments of the present invention described above are intended to be merely exemplary. Those skilled in the art will recognize, or be able to ascertain using no more than routine experimentation, numerous equivalents to the specific procedures described herein. All such equivalents are considered to be within the scope of the present invention and are covered by the following claims.

It is further contemplated that any embodiment or implementation of the disclosure manifested above as a system or method may include at least a portion of any other embodiment or implementation described herein. Those having skill in the art will appreciate that there are various embodiments or implementations by which systems and methods described herein can be implemented, and that the implementation will vary with the context in which an embodiment of the disclosure is deployed.

Conclusion

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. A composition, comprising:
a peroxisome proliferator activated receptor gamma (PPARγ) antagonist that is administered to an individual, where the individual has been administered a ketogenic diet.

2. The composition of claim 1, wherein the PPARγ antagonist includes GW9662.

3. The composition of claim 1, wherein the PPARγ antagonist includes pioglitazone.

4. The composition of claim 1, wherein the PPARγ antagonist is configured to be administered orally.

5. The composition of claim 1, wherein the PPARγ antagonist is configured to be administered intravenously.

6. The composition of claim 1, wherein the PPARγ antagonist is configured to be administered exogenously.

7. The composition of claim 1, wherein the PPARγ antagonist includes a PPARγ2 antagonist.

8. A method for administering an anti-seizure composition, comprising:
administering a ketogenic diet to an individual; and
concurrently administering a therapeutically effective dose of a peroxisome proliferator activated receptor gamma (PPARγ) antagonist to the individual.

9. The method of administering an anti-seizure composition of claim 8, wherein the PPARγ antagonist includes GW9662.

10. The method of administering an anti-seizure composition of claim 8, wherein the PPARγ antagonist includes pioglitazone.

11. The method of administering an anti-seizure composition of claim 8, wherein the PPARγ antagonist is configured to be administered orally.

12. The method of administering an anti-seizure composition of claim 8, wherein the PPARγ antagonist is configured to be administered intravenously.

13. The method of administering an anti-seizure composition of claim 8, wherein administering the PPARγ antagonist includes administering the antagonist exogenously.

14. The method of administering an anti-seizure composition of claim 8, wherein administering the PPARγ antagonist includes administering a PPARγ2 antagonist.

15. The method of administering an anti-seizure composition of claim 8, wherein administering the anti-seizure composition reduces seizure frequency.

16. A method for treating a seizure, comprising:
administering a therapeutically effective dose of a peroxisome proliferator activated receptor gamma (PPARγ) antagonist to an individual on a ketogenic diet and suffering from seizures.

17. The method of treating a seizure of claim 16, wherein the seizure is an epileptic seizure.

18. The method of treating a seizure of claim 16, wherein the seizure is a refractory seizure that does not respond to seizure medication.

19. The method of treating a seizure of claim 16, wherein the PPARγ antagonist includes GW9662.

20. The method of treating a seizure of claim 16, wherein the PPARγ antagonist includes pioglitazone.
